(19) United States
(12) Patent Application Publication
(10) Pub. No.: US 2025/0161390 A1
(43) Pub. Date: May 22, 2025

(54) METHOD FOR PREPARING RED GINSENG EXTRACT WHICH HAS HIGH CONTENT OF GINSENOSIDE AND RED GINSENG EXTRACT PREPARED BY USING SAME

(71) Applicant: Haein Co., Ltd., Yeongju-si (KR)

(72) Inventor: Lincoln SHIN, Yeongju-si (KR)

(73) Assignee: Haein Co., Ltd., Yeongju-si (KR)

(21) Appl. No.: 18/677,187

(22) Filed: May 29, 2024

(30) Foreign Application Priority Data
Nov. 20, 2023 (KR) 10-2023-0160399

(51) Int. Cl. A61K 36/258; A61K 41/17; A61L 2/00; A61L 2/18

(52) U.S. Cl. CPC A61K 36/258; A61K 41/17; A61L 2/0047; A61L 2/183; A61K 2236/13; A61K 2236/15; A61K 2236/331; A61K 2236/37; A61K 2236/39; A61K 2236/51; A61K 2236/53; A61L 2202/11; A61L 2202/21

(57) ABSTRACT

The present invention relates to a method for preparing red ginseng extract and red ginseng extract prepared by using the same and, more specifically, to a method for preparing red ginseng extract which has a high content of ginsenoside, is hygienic, has a good flavor, easy to take with a high sweetness the ginseng through a trimming step, a washing and sterilization step, a steaming step, a drying step, and an extraction step, and to the red ginseng extract manufactured by the same.

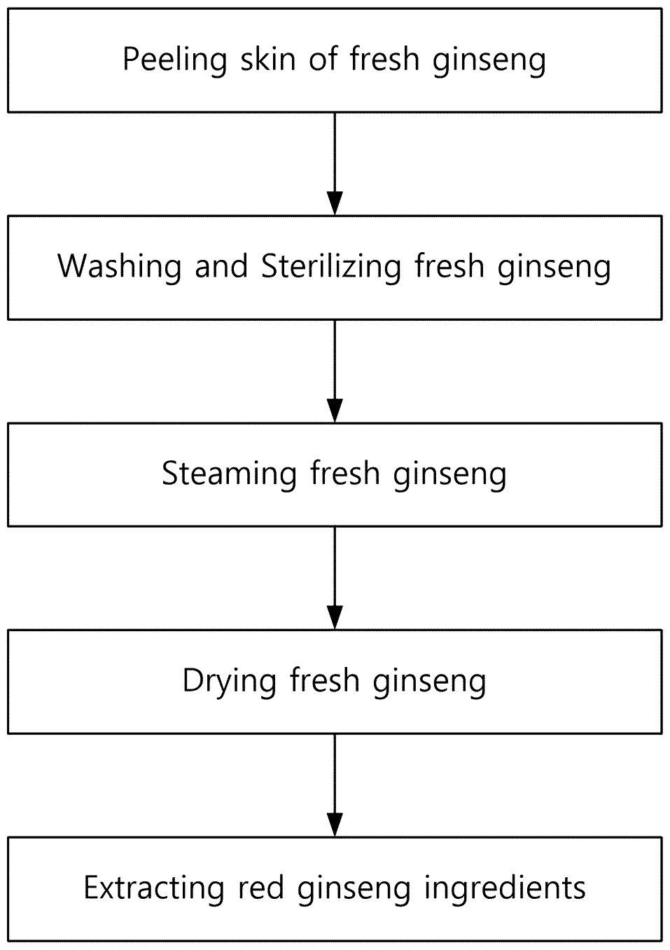

### CROSS-REFERENCE TO RELATED APPLICATION

This application claims priority to and the benefit of Korean Patent Application No. 10-2023-0160399, filed on Nov. 20, 2023, the disclosure of which is incorporated herein by reference in its entirety.

### BACKGROUND

### 1. Field of the Invention

The present invention relates to a method for preparing red ginseng extract and red ginseng extract prepared by using the same and, more specifically, to a method for preparing red ginseng extract which has a high content of ginsenoside, is hygienic, has a good flavor, has a high sweetness, and easy to take, and to the red ginseng extract manufactured by the same.

### 2. Discussion of Related Art

As national income increases and the elderly population increases rapidly, the consumption of ginseng products, which are health supplements, is on the rise. Recently, health supplements of ginseng are being distributed in various forms, but among them, the consumption of ginseng extracts (pouches) that can be easily taken by consumers is increasing.

Korean ginseng, a plant taxonomic plant belonging to the ginseng genus Panax of Araliaceae, has long been used as an important medicinal ingredient in oriental medicine. Scientific research on ginseng began in 1854 when Garriques from the United States separated an amorphous glycoside mixture from Western ginseng and named it ‘panaquilon’, but full-scale research began in 1957 when Brekhman, a Soviet pharmacologist, announced that the active ingredient of ginseng was saponin, and in the 1960s, research on ginseng was accelerated as the chemical structure of ginseng saponin was clearly revealed by Shibata from Japan and others. Studies on the pharmacological and clinical effects of ginseng known to date have reported anti-diabetic and anti-oxidative effects, prevention of arteriosclerosis and hypertension, liver function activity, anti-fatigue and stress effects, anti-aging effects, and anti-inflammatory activities.

Korean ginseng is generally classified into white ginseng and red ginseng, and white ginseng is obtained by drying fresh ginseng as it is, and red ginseng is obtained by steaming and drying the fresh ginseng, which is accompanied by various chemical changes such as saponin modification, amino acid change, and brown change during the manufacturing process. Particularly, saponins such as ginsenoside Rb1, Rb2, Rg2, Rg3, Rh1, Rh2 and the like, which are unique ingredients of red ginseng that do not exist in ginseng, are newly generated by the heat generated in the process of preparing red ginseng, and such red ginseng-specific saponins are known to have cancer prevention activity, cancer cell growth inhibition activity, blood pressure lowering activity, brain nerve cell protection and learning ability improvement activity, antithrombotic activity, antioxidant activity and the like, and thus it is expected to have excellent pharmacological effects of red ginseng.

Most of the red ginseng is taken after being extracted at high temperatures for a long time, but the extraction temperature and extraction time to increase functionality have not been established. The amount of ginsenoside of red ginseng varies according to the extraction temperature and the extraction time by type, and most domestic producers producing the red ginseng extract are extracting the red ginseng according to an empirical method in a state that scientific research on the change of useful saponin and physical properties of the red ginseng extract according to the extraction temperature or the extraction time is insufficient.

### SUMMARY OF THE INVENTION

The problem to be solved by the present invention is to provide a method for preparing red ginseng extract which has a high content of ginsenoside, is hygienic and has a good flavor, easy to take with a high sweetness.

Another problem to be solved by the present invention is to provide a red ginseng extract prepared by the method.

The objects of the present invention are not limited to the above-mentioned technical objects, and other unmentioned technical objects will be clearly understood by the skilled person in the art from the following description.

According to an aspect of the present invention, there is provided a method for preparing red ginseng extract, the method including: a step of peeling the skins of 5-6 years old fresh ginseng; a step of washing the peeled fresh ginseng with a 5 wt % ethanol aqueous solution; a step of sterilizing washing which washing the washed fresh ginseng with ozone water while irradiating ultraviolet rays thereto, and then further irradiating ultraviolet rays thereto; a step of washing the sterilized and washed fresh ginseng with distilled water; a step of steaming the washed fresh ginseng with a vapor of 95-120° C. generated by heating a natural medicinal material mixture including 3-5 wt % of licorice, 0.5-2 wt % of hydrangea leaves, 1-3 wt % of puerariae radix, 1-3 wt % of Angelica gigas, 2-5 wt % of Kalopanax septemlobus, 0.5-2 wt % of skin of Citrus reticulata Blanco, and a remainder of rice water; a step of drying the steamed fresh ginseng in sea breeze for 1-3 days; a step of drying the dried fresh ginseng with hot air of 75-85° C. and relative humidity of 50-60% for 12-15 hours; a step of preparing red ginseng by irradiating the hot air-dried fresh ginseng with far infrared rays having a wavelength of 10-1000 μm for 2-5 hours; a step of cutting the red ginseng into main root, supporting roots, and fine roots, and pressing each of them at a pressure of 5-20 N/cm² for 10-40 minutes; a step of adding the main roots, the supporting roots, and the fine roots into the mineral water mixed with 10-20 wt % of deep sea water and heating the mineral water to obtain each extract of the main roots, the supporting roots, and the fine roots, respectively; a step of mixing extracts of the main roots, the supporting roots, and the fine roots and filtering the mixture in a mesh sieve of 35-140 mesh to obtain a first extract, re-introducing the filtered red ginseng residue into mineral water in which 10-20 wt % of deep sea water is mixed, and heating the mixture at 80-85° C. for 12-18 hours to obtain a second extract; and a step of mixing the primary extract and the secondary extract, wherein the extract of the main roots obtained by heating the main roots at 90-95° C. for 25-30 hours, the extract of the supporting roots is obtained by heating the supporting roots at 85-90° C. for 15-25 hours, and the extract of the fine roots is obtained by heating the fine roots at 75-85° C. for 12-15 hours.

The ultraviolet rays irradiated in the sterilizing and washing step may irradiate UV-C having a wavelength of 280-100 nm in the washing of the ozone water, and irradiate UV-B having a wavelength of 315-280 nm after the washing.

In the sterilization and washing step, ultraviolet rays irradiated during the washing of the ozone water may be irradiated in the form of pulses.

Meanwhile, the heating of the main roots, the supporting roots, and the fine roots and/or the heating of the filtered red ginseng residue may be performed under a 2 to 20 atmospheric pressure.

A red ginseng extract according to an embodiment of the present invention for solving the above problem is prepared by the method.

Details of other embodiments are included in the detailed description.

### BRIEF DESCRIPTION OF THE DRAWINGS

The FIGURE is a flowchart illustrating a step of preparing a red ginseng extract according to an embodiment of the present invention.

### DETAILED DESCRIPTION OF EXEMPLARY EMBODIMENTS

Advantages and features of the present invention, and methods of achieving the same will become apparent with reference to embodiments described in detail below. However, the present invention is not limited to the embodiments described below, but may be implemented in various different forms, and the embodiments are provided so that the disclosure of the present invention is complete and the scope of the present invention is fully known to a person having ordinary skill in the art to which the present invention belongs, and the present invention is defined only by the scope of the claims.

The terminology used herein is for the purpose of describing embodiments and is not intended to be limiting of the present invention. In this specification, “and/or” includes each of the mentioned items and all combinations of one or more. In addition, a singular form includes a plural form unless specifically mentioned in the text. The terms “comprises” and/or “comprising” used in the specification do not exclude the presence or addition of one or more other elements in addition to the mentioned elements. The numerical range represented using “-” or “to” represents a numerical range including the values described before and after it as a lower limit and an upper limit, respectively, unless otherwise stated. “Approximately” or “about” means a value or a numerical range within 20% of the value or numerical range described later.

In addition, in describing the feature element of the embodiment of the present invention, terms such as first, second, A, B, (a), (b), and the like may be used. The term is used only to distinguish the feature element from other feature elements, and the nature, sequence, or order of the corresponding feature element is not limited by the term.

Unless otherwise defined, all terms (including technical and scientific terms) used herein have the same meaning as commonly understood by one of ordinary skill in the art to which the inventive concept belongs. In addition, terms defined in commonly used dictionaries are not interpreted ideally or excessively unless they are clearly specifically defined.

In describing the embodiments of the present invention, when it is determined that the detailed description of the related known feature or function interferes with the understanding of the embodiments of the present invention, the detailed description thereof will be omitted.

Hereinafter, embodiments of the present invention will be described in detail.

Referring to the Figure, the method for preparing red ginseng extract according to an embodiment of the present invention may be largely divided into a trimming step, a washing and sterilization step, a steaming step, a drying step, and an extraction step.

### Trimming Step

It is a step of securing 5-6 years old fresh ginseng and peeling the skins before washing, and it is possible to easily remove rotten parts, large foreign substances or impurities.

### Washing and Sterilization Step

The trimmed fresh ginseng is pre-washed with 5 wt % ethanol aqueous solution, is main-washed with ozone water while being irradiated with ultraviolet rays, and is then further irradiated with ultraviolet rays. It is possible to sterilize while washing the fresh ginseng with ozone water, and to add the sterilization effect of the fresh ginseng by irradiating ultraviolet rays.

In some embodiments, UV-C having a wavelength of 280-100 nm may be irradiated when washing with ozone water, and UV-B having a wavelength of 315-280 nm may be irradiated for 10 minutes-2 hours after washing. The UV-C may promote the formation of ozone to enhance the sterilization effect of ozone water, and the UV-B may promote the decomposition of ozone to remove ozone remaining after washing the ozone water.

In some embodiments, ultraviolet rays irradiated during washing of ozone water may be irradiated in the form of pulses. The “pulse irradiation” refers to irradiation of ultraviolet rays to have a predetermined period, and specifically, to repeating irradiation of ultraviolet rays and non-irradiation at a predetermined period. In an exemplary embodiment, the UV light may be pulse-irradiated to repeat irradiation and non-irradiation with an irradiation period of 1 to 10 minutes and a non-irradiation period of 10 sec to 3 minutes, but the present invention is not limited thereto. The surface of the fresh ginseng can be minimized from being damaged by ultraviolet rays while maintaining the sterilization effect through the irradiation of ultraviolet rays.

The fresh ginseng sterilized and washed with ozone water and ultraviolet rays is post-washed with distilled water to remove residues, thereby completing the washing.

### Steaming Step

The washed fresh ginseng is steamed for 3.5-5 hours with steam at 95-120° C. generated by heating the natural medicine mixed water. The mixed water of the natural medicine is a mixture including various medicines and natural materials, and includes 3-5 wt % of licorice, 0.5-2 wt % of hydrangea leaves, 1-3 wt % of puerariae radix, 1-3 wt % of angelica gigas, 2-5 wt % of Kalopanax septemlobus, 0.5-2 wt % of skin of Citrus reticulata Blanco, and a remainder of rice water. The fresh ginseng which is steamed for 3.5 hours or more with the mixed water of the natural medicine may have good flavor and softness, but the steaming for 5 hours or more may be excessive heat waste compared to the effect.

### Drying Step

The steamed fresh ginseng goes through the steps of sea breeze drying, hot air drying, and far-infrared drying. First, the organic layer is dried for 1-3 days by using sea breeze on the shore, and then dried for 12-15 hours by using hot air at 75-85° C. and a relative humidity of 50-60%, and finally, the organic layer is dried by irradiating far infrared rays having a wavelength of 10-1000 μm for 2-5 hours. The dried fresh ginseng is referred to as red ginseng.

### Extraction Step

Red ginseng prepared by trimming, washing, steaming, and drying fresh ginseng is cut into main roots, supporting roots, and fine roots. The main root, the supporting root, and the fine root are named according to the part of red ginseng, and are referred to as the main root, the supporting root, and the fine root in order from the top to the bottom. In the present invention, the term “main root, supporting root, and fine root” may refer to a concept including a peripheral region which is not referred to as the main root, supporting root, and fine root within a reasonable range.

The cut main roots, supporting roots, and fine roots are pressed for 10-40 minutes at a pressure of 5-20 N/cm², respectively. The surface area can be increased by pressurizing red ginseng and inducing deformation, and thus the discharge of active ingredients such as ginsenosides can be promoted. There is no particular limitation on the pressing means, and equipment such as a press may be used, or manpower may be used directly.

The pressed main roots, supporting roots, and fine roots are separately introduced into mineral water mixed with 10-20 wt % of deep sea water, respectively, and then heated by an extractor to obtain and mix each extract of the main roots, supporting roots, and fine roots. That is, extracts are prepared for each of the main roots, supporting roots, and fine roots, and then these are mixed with each other.

In general, as red ginseng is heated at a high temperature for a long time, the extraction amount of ginsenoside decreases, but the sweetness tends to increase. However, in the case of the main roots, supporting roots, and fine roots, the total content of saponin and ginsenoside is different from each other, and thus, if the heating temperature and time of the main roots, supporting roots, and fine roots are different from each other, both the extraction amount and the sweetness of ginsenoside can be increased. Specifically, since the content of total saponin and ginsenoside is highest in the fine roots and lowest in the main roots, the heating temperature and time of the fine roots may be lowest and the heating temperature and time of the main roots may be highest. In particular, the optimum heating temperature and time conditions for each of the main roots, supporting roots, and fine roots are combined to maximize the extraction amount of ginsenoside and improve the sweetness.

In some embodiments, the extract of the main root is obtained by heating the main roots to 90-95° C. for 25-30 hours, the extract of the supporting root is obtained by heating the supporting roots to 85-90° C. for 15-25 hours, and the extract of the fine root is obtained by heating the fine roots to 75-85° C. for 12-15 hours. The mixture of each extract prepared under such conditions may have high sweetness while having excellent ginsenoside content.

The extract obtained by heating the main roots, supporting roots, and fine roots of red ginseng is mixed with each other and filtered through a sieve of 35-140 mesh sieve, thereby obtaining a first extract from which large residues of red ginseng are filtered out. The filtered red ginseng residues are added to mineral water mixed with 10-20 wt % deep sea water and heated at 80-85° C. for 12-18 hours to obtain a secondary extract. If the concentration of an active ingredient such as ginsenoside in the extract increases, the extraction can be slowed down due to osmotic pressure even if the active ingredient remains in red ginseng, and thus the amount of the active ingredient to be extracted can be maximized by recovering the residuals of the red ginseng which is first extracted and then injecting the residuals into a new hot water bath for secondary extraction.

Further, after the primary extract is cooled to 5-10° C., the cooled primary extract may be secondarily filtered through a sieve of 15-50 mesh. Residues may be additionally filtered through cooling and filtration processes, and the secondarily filtered residues may be dried and pulverized to mix with the primary extract. Accordingly, it is possible to prepare a red ginseng liquid which can be conveniently drunk while minimizing the loss of active ingredients.

In some embodiments, the pressure in the extractor may be maintained at 2-20 atmospheric pressure, preferably 3-10 atmospheric pressure, when the red ginseng is heated and extracted to improve the quality of the red ginseng extract, but is not limited thereto.

The red ginseng extract of the present invention may be obtained by mixing the primary extract and the secondary extract and further passing a predetermined filtration or purification step and a treatment step. The red ginseng extract can be delivered to a consumer through a predetermined packaging step.

Here, the weight and temperature of the primary extract and the secondary extract may be measured. For example, the primary extract may be cooled to 10° C. and the secondary extract may be heated to 85° C. In this case, the two extracts are mixed and then heated at an optimal temperature and time to minimize the loss of the active ingredient and uniformly mix the two extracts.

The target temperature (unit: ° C.) is calculated using the following equation in consideration of the temperature and weight of the two extracts.

$$Target temperature = ( W 1 * T 2 + W 2 * T 1 ) / ( W 1 + W 2 )$$

(where W1 is the weight of the primary extract, W2 is the weight of the secondary extract, T1 is the temperature of the primary extract, and T2 is the temperature of the secondary extract. The unit of weight is t (tons) and the unit of temperature is ° C.)

In addition, the heating time (unit: minute) is calculated using the following equation.

$$Heating time = \vert T 1 - T 2 \vert * ( W 1 + W 2 )$$

The red ginseng extract according to an embodiment of the present invention is prepared by the above-described preparation method, and is a high-quality red ginseng extract which is hygienic and has good flavor, has a high content of active ingredients such as ginsenosides, and high sweetness.

Hereinafter, the present invention will be described through Preparation Examples and Experimental Examples, but it is obvious that the effects of the present invention are not limited by the following Experimental Examples.

### Preparation Example: Preparation of a Red Ginseng Extract

### Embodiment

Fresh 5-6 year old ginseng was purchased and the skin and foreign substances were removed. Fresh ginseng was washed with 5 wt % aqueous ethanol solution, then with ozonated water while being irradiated with UV-C, and then further irradiated with UV-B for about 20 minutes, finished with distilled water washing. Fresh ginseng was steamed for about 4 hours by heating mixed water of natural medicinal materials including 4 wt % of licorice, 1 wt % of hydrangea leaves, 2 wt % of puerariae radix, 2 wt % of Angelica gigas, 3 wt % of Cinnamon tree, 1 wt % of dermis, and the remaining amount of rice water. The steamed fresh ginseng was dried in a sea breeze for about one day, dried in hot air at 75-80° C. and a relative humidity of 50-55% for 12 hours, and then irradiated with far-infrared rays for 2 hours to obtain red ginseng.

Red ginseng was cut into the main roots, the supporting roots, and the fine roots, pressed with a press at a pressure of 10 N/cm² for about 15 minutes, separated into several extractors containing 10 wt % of deep sea water and mineral water, and heated at 95° C. for 30 hours, at 85° C. for 20 hours, and at 75° C. for 12 hours to obtain each extract. The respective extracts were mixed with each other and filtered through a sieve of 45 mesh to obtain a first extract, the filtered red ginseng residue was put again into mixed water of 10 wt % deep sea water, and heated at 85° C. for 18 hours to obtain a second extract, the first extract was cooled to 10° C., filtered through a sieve of 20 mesh to dry and pulverize the filtrate, and then mixed with the first extract. A red ginseng extract was prepared by mixing 2 t of a primary extract at 10° C. and 1 t of a secondary extract at 85° C., heating at a temperature of 60° C. for 225 minutes, and then purifying the same.

### Comparative Example 1

A red ginseng extract was prepared in the same manner as in Example 1 except that mineral water was heated for about 4 hours to steam fresh ginseng.

### Comparative Example 2

A red ginseng extract was prepared in the same manner as in Example 1, except that red ginseng was introduced into a barrel without being separated into a main root, a supporting root, and a fine root, and then heated at 85° C. for 20 hours.

### Comparative Example 3

A red ginseng extract was prepared in the same manner as in Example 1 except that the primary extract was cooled and refiltered.

### Comparative Example 4

A red ginseng extract was prepared in the same manner as in Example 1 except that the primary extract and the secondary extract were mixed and then reheated.

### Experimental Example: Sensory Evaluation of a Red Ginseng Extract

Five evaluators were arbitrarily selected regardless of gender and age to perform sensory evaluation on the prepared red ginseng extract. The viscosity, taste, and aroma preference of the evaluators with respect to the red ginseng extract were evaluated as a perfect score of 5, and then an average value was calculated, and the results are shown in Table 1 below.

TABLE 1

| | Viscosity | Taste | Aroma |
|---|---|---|---|
| | preference | preference | preference |
| Embodiment | 4.4 | 4.2 | 3.6 |
| Comparative | 4 | 4 | 2.8 |
| Example 1 | | | |
| Comparative | 3 | 3.6 | 3.4 |
| Example 2 | | | |
| Comparative | 2.8 | 3.5 | 3.4 |
| Example 3 | | | |
| Comparative | 2.5 | 3.2 | 3.1 |
| Example 4 | | | |

In general, evaluators showed higher viscosity preference as the red ginseng extract was thicker, and the less bitter the taste, the higher the taste preference. As shown in Table 1, the red ginseng extract prepared by the method of the present invention had the highest preference in all of viscosity, taste, and aroma.

The red ginseng extract prepared according to embodiments of the present invention has a high level of ginsenosides such as Rb1, Rb2, and Rg3, and thus the content thereof is excellent, is hygienic, has a good flavor, and easy to take with a high sweetness.

The effects according to the embodiments of the present invention are not limited by the contents exemplified above, and more various effects are included in the present specification.

Although the embodiments of the present invention have been described above, these are merely examples and the present invention is not limited thereto, and those skilled in the art will appreciate that various modifications and applications that are not illustrated above may be made without departing from the essential characteristics of the embodiments of the present invention. For example, each feature element specifically shown in embodiments of the present invention may be modified and embodied. Further, the differences related to the modifications and applications should be interpreted as being included in the scope of the present invention defined in the appended the claims.

What is claimed is:

1. A method of preparing a red ginseng extract, the method comprising:
washing the peeled fresh ginseng with a 5 wt % ethanol aqueous solution;
sterilizing washing which washing the washed fresh ginseng with ozone water while irradiating ultraviolet rays thereto, and then further irradiating ultraviolet rays thereto;
washing the sterilized and washed fresh ginseng with distilled water;
steaming the washed fresh ginseng with a vapor of 95-120° C. generated by heating a natural medicinal material mixture including 3-5 wt % of licorice, 0.5-2 wt % of hydrangea leaves, 1-3 wt % of puerariae radix, 1-3 wt % of Angelica gigas, 2-5 wt % of Kalopanax septemlobus, 0.5-2 wt % of skin of Citrus reticulata Blanco, and a remainder of rice water;
drying the steamed fresh ginseng in sea air for 1-3 days;
drying the dried fresh ginseng with hot air of 75-85° C. and 50-60% relative humidity for 12-15 hours;
preparing red ginseng by irradiating the hot air-dried fresh ginseng with far infrared rays having a wavelength of 10-1000 μm for 2-5 hours;
cutting the red ginseng into main roots, supporting roots, and fine roots, and pressing each of them at a pressure of 5-20 N/cm² for 10-40 minutes;
adding the main roots, the supporting roots, and the fine roots into the mineral water mixed with 10-20 wt % of deep sea water and heating the mineral water to obtain each extract of the main roots, the supporting roots, and the fine roots, respectively;
mixing extracts of the main roots, the supporting roots, and the fine roots and filtering the mixture in a mesh sieve of 35-140 mesh to obtain a first extract, re-introducing the filtered red ginseng residue into mineral water in which 10-20 wt % of deep sea water is mixed, and heating the mixture at 80-85° C. for 12-18 hours to obtain a second extract; and
mixing the primary extract and the secondary extract,
wherein the extract of the main roots obtained by heating the main roots at 90-95° C. for 25-30 hours, the extract of the supporting roots is obtained by heating the supporting roots at 85-90° C. for 15-25 hours, and the extract of the fine roots is obtained by heating the fine roots at 75-85° C. for 12-15 hours,
wherein the obtaining primary extract and secondary extract comprises:
cooling the primary extract to 5-10° C.;
secondarily filtering the cooled primary extract with a mesh sieve of 15-50 mesh;
drying and pulverizing the secondarily filtered residue; and
mixing the dried and pulverized residue with the primary extract,
wherein the mixing of the primary extract and the secondary extract comprises:
measuring the weight and temperature of the primary extract and the secondary extract;
calculating the target temperature (unit: ° C.) using the following Equation:
$$Target temperature = ( W 1 * T 2 + W 2 * T 1 ) / ( W 1 + W 2 )$$
(where W1 is the weight of the primary extract, W2 is the weight of the secondary extract, T1 is the temperature of the primary extract, and T2 is the temperature of the secondary extract, The unit of weight is t (tons) and the unit of temperature is ° C.);
calculating the heating time (unit: minutes) using the following equation; and
$$h eating time = \vert T 1 - T 2 \vert * ( W 1 + W 2 )$$
heating a mixture of the primary extract and the secondary extract at the calculated target temperature for the calculated heating time.

2. The method for preparing a red ginseng extract of claim 1,
wherein the ultraviolet rays irradiated in the sterilization and washing step are irradiated with UV-C having a wavelength of 280-100 nm during the washing of the ozone water and irradiated with UV-B having a wavelength of 315-280 nm after the washing.

3. The method of preparing a red ginseng extract of claim 1,
wherein in the sterilization and washing step, the ultraviolet rays irradiated when the ozone water is washed are irradiated in a pulse form.

4. The method for preparing a red ginseng extract of claim 1,
wherein the heating the main roots, supporting roots, and the fine roots and/or the heating the filtered red ginseng residue are performed under a pressure of 2-20 atmospheric pressure.

5. A red ginseng extract prepared by the method of claim 1.
